(19) United States
(12) Patent Application Publication
(10) Pub. No.: US 2015/0080628 A1
(43) Pub. Date: Mar. 19, 2015

(54) CATALYST AND PROCESS FOR THE CO-DIMERIZATION OF ETHYLENE AND PROPYLENE

(71) Applicant: Eastman Chemical Company, Kingsport, TN (US)

(72) Inventors: David William Norman, Cary, NC (US); Joy Lynn Laningham, Erwin, TN (US)

(73) Assignee: Eastman Chemical Company, Kingsport, TN (US)

(21) Appl. No.: 14/031,697

(22) Filed: Sep. 19, 2013

(51) Int. Cl. C07C 2/34

(52) U.S. Cl. CPC C07C 2/34
USPC 585/513; 502/117

(57) ABSTRACT

Disclosed are novel catalyst solutions comprising an organic complex of nickel, an alkyl aluminum compound, a solvent, and a phosphine compound, that are useful for the preparation of butenes, pentenes and hexenes by the co-dimerization or cross-dimerization of ethylene and propylene. Also disclosed are processes for the dimerization of ethylene and propylene that utilize these catalyst solutions. The catalyst systems described herein demonstrate that, depending on the choice of phosphine compound used with the catalytically active nickel, it is indeed possible to lower the concentration of hexene olefins relative to butenes and pentenes, even in the presence of excess propylene. The selectivity to the linear or branched pentene product can also be controlled by the selection of the phosphine compound. The catalyst solutions may be used with mixtures of olefins.

### FIELD OF THE INVENTION

The present invention generally relates to a novel catalyst system and process for the co-dimerization of ethylene and propylene to yield product mixtures of butenes, pentenes, and hexenes. More specifically, this invention pertains to catalyst solutions that comprise an organic complex of nickel, alkyl aluminum compound, solvent, and phosphine compound, and dimerization processes that use these catalyst solutions.

### BACKGROUND

Olefin or alkene products, such as ethylene and propylene, are vital feedstocks for the chemical industry, serving as precursors for numerous chemical derivatives and functional materials. Typically, these valuable starting materials are derived from cracking petroleum feedstocks and natural gas liquids (‘NGLs’). Recent expansion of the shale gas industry, particularly in North America, has lowered the cost of NGLs to such an extent that many industrial cracking facilities are shifting to this lighter feedstock to produce olefin mixtures. Separating mixtures of ethylene and propylene requires costly unit operations involving successive compressions and distillations. Processes that utilize mixtures of ethylene and propylene without this separation procedure would undoubtedly lower this feedstock cost and allow downstream products to be more competitively priced.

Catalytic co-dimerization or cross-dimerization of ethylene and propylene is one such technology that could exploit a low cost, unrefined mixture of ethylene and propylene. For example, a catalyst with the ability to co-dimerize an ethylene molecule with propylene to form pentenes (‘C5s’) can also dimerize ethylene to form butenes (‘C4s’) and propylene to form hexenes (‘C6s’). The C4, C5, and C6 olefins derived from ethylene/propylene co-dimerization are useful particularly when coupled with conventional dehydrogenation technology. Of these products, however, there is often more demand for the C4 and C5 over the C6 products. For example, linear butenes (1-butene and 2-butene) can be dehydrogenated to butadiene, linear pentenes (1-pentene and 2-pentene) can be dehydrogenated to 1,3-pentadiene (piperylene), and branched pentenes (2-methyl-2-butene and 2-methyl-1-butene) can be dehydrogenated to isoprene.

The product distribution from co-dimerization reactions, however, is frequently difficult to control and optimize. Given the latent value of butenes and pentenes, there is a need for catalysts that can minimize the amount of C6 olefins and maximize both C4s and C5s produced in ethylene/propylene co-dimerizations, especially under reaction conditions which inherently favor the production of C6 olefins such as, for example, when feedstocks containing an excess of propylene are used. There is a further need for catalysts that can control the relative amount of linear and branched C5s from ethylene/propylene co-dimerization processes.

### BRIEF SUMMARY

We have discovered novel catalyst solutions that enable the selective production of C4 and C5 olefins over C6 olefins through the dimerization of ethylene and propylene.

In a first embodiment, the present invention is a catalyst solution comprising: (i) an organic complex of nickel; (ii) an alkyl aluminum compound; (iii) a solvent; and (iv) at least one phosphine compound having the formula: PR¹R²R³ wherein R¹ and R² each are independently selected from the group consisting of t-butyl, 2-pyridyl, 2,6-dimethoxyphenyl, o-tolyl, cyclohexyl, phenyl, butyl, and adamantyl; and wherein R³ is selected from the group consisting of 2-pyridyl, 2,6-dimethoxyphenyl, o-tolyl, 2′,4′,6′-triisopropylbiphenyl, 2′-(N,N-dimethylamino)biphenyl, adamantyl, 1-(2,4,6-trimethyl-phenyl)-1H-imidazole, and 1,2,3,4,5-pentaphenyl-1′-ferrocene.

In a second embodiment, the present invention is a catalyst solution comprising: (i) an allylnickel halide catalyst; (ii) an alkyl aluminum compound; (iii) a solvent; and (iv) at least one phosphine compound selected from the following structures:

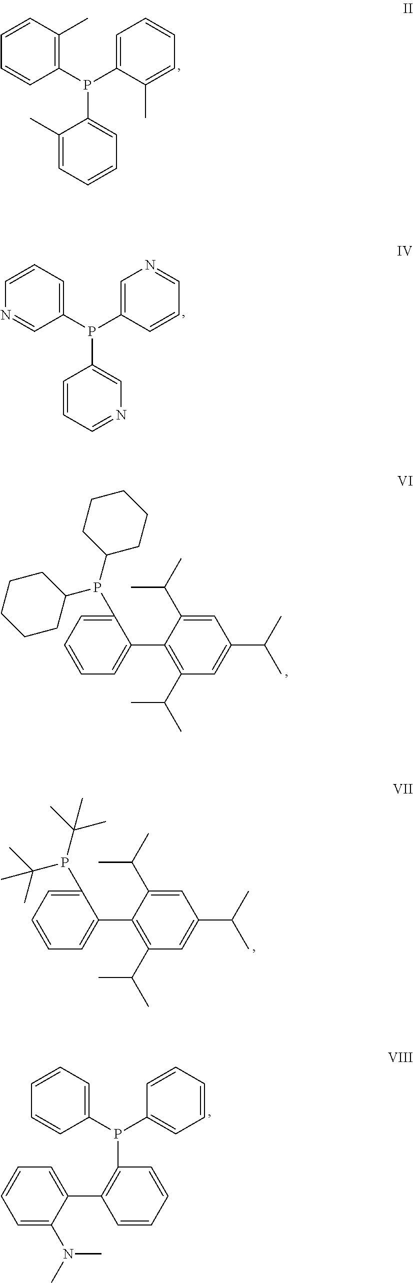

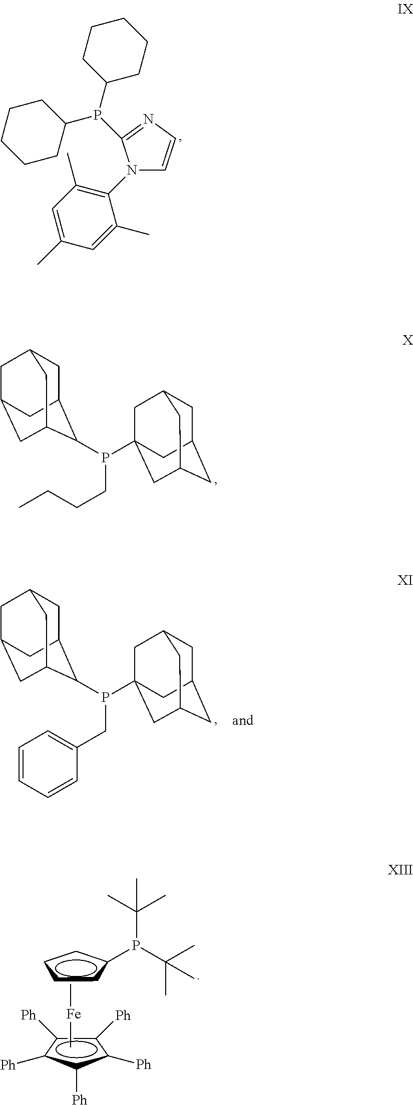

In a third embodiment, the present invention is an ethylene and propylene co-dimerization process comprising contacting ethylene and propylene under elevated pressure with a catalyst solution comprising: (i) an allylnickel halide catalyst; (ii) an alkyl aluminum compound; (iii) a solvent; and (iv) at least one phosphine compound selected from the following structures:

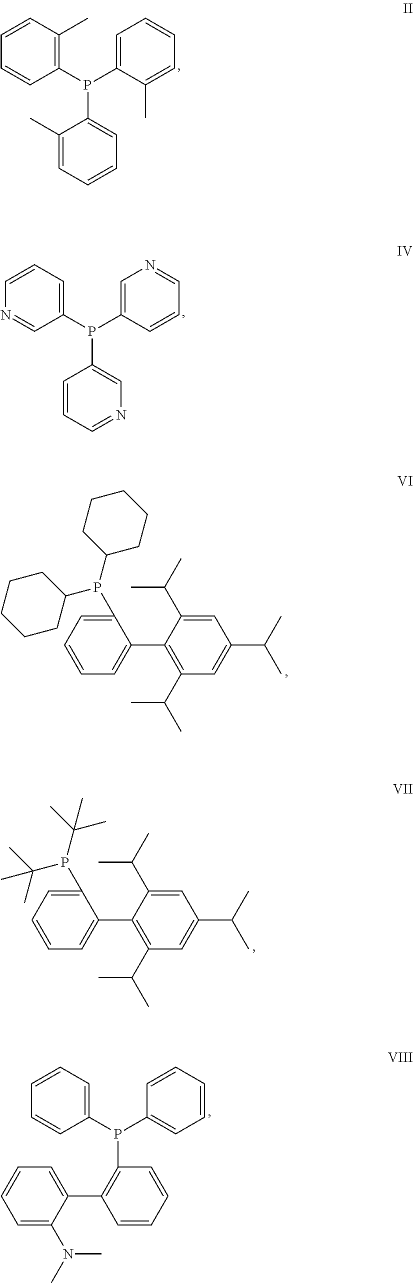

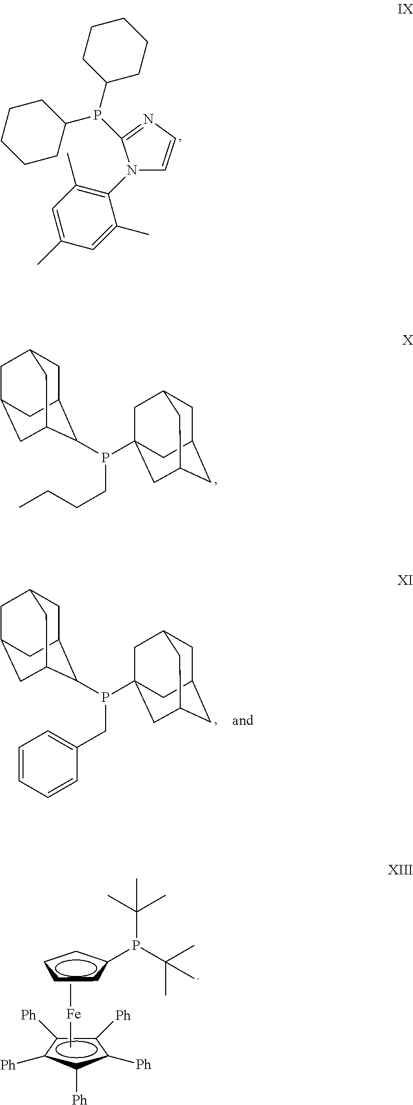

The catalyst systems described herein demonstrate that, depending on the choice of phosphine compound modifier used with the catalytically active nickel, it is indeed possible to lower the concentration of C6 olefins relative to C4s and C5s, even in the presence of excess propylene. Moreover, it has been discovered that certain phosphine compounds can significantly affect the amount of branched C5 products relative to linear C5s.

### DETAILED DESCRIPTION

It has been discovered that ethylene/propylene co-dimerization catalyst solutions can be prepared from a variety of phosphine compounds combined with an organic complex of nickel, and an alkyl aluminum compound in a solvent. Employing these co-dimerization catalyst solutions can provide a lower concentration of C6 olefins relative to C4s and C5s, even in the presence of excess propylene. Some of these co-dimerization catalyst solutions have been discovered to significantly affect the amount of branched C5 products relative to linear C5s.

In a first embodiment, the present invention is a catalyst solution comprising: (i) an organic complex of nickel; (ii) an alkyl aluminum compound; (iii) a solvent; and (iv) at least one phosphine compound having the formula: PR¹R²R³ wherein R¹ and R² each are independently selected from the group consisting of t-butyl, 2-pyridyl, 2,6-dimethoxyphenyl, o-tolyl, cyclohexyl, phenyl, butyl, and adamantyl; and wherein R³ is selected from the group consisting of 2-pyridyl, 2,6-dimethoxyphenyl, o-tolyl, 2′,4′,6′-triisopropylbiphenyl, 2′-(N,N-dimethylamino)biphenyl, adamantyl, 1-(2,4,6-trimethyl-phenyl)-1H-imidazole, and 1,2,3,4,5-pentaphenyl-1′-ferrocene.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

The organic complex of nickel component of the catalyst solution comprises a bis(triphenylphosphine)dicarbonylnickel complex or a π-allyl nickel halide complex. Certain embodiments have π-allyl moieties from 3 up to and including 12 carbon atoms. Examples of some π-allyl nickel halides include, but are not limited to, methylallylnickel chloride, methylallylnickel bromide, methylallylnickel iodide, allylnickel chloride, allylnickel bromide, allylnickel iodide, crotylnickel chloride, ethylallylnickel chloride, cyclopentylallylnickel chloride, cyclooctenylnickel chloride, cinnamylnickel bromide, phenylallylnickel chloride, cyclohexenylnickel bromide, cyclodecenylnickel chloride, and/or dimers thereof. Examples of some suitable π-allyl nickel halides dimers are methylallylnickel chloride dimer, methylallylnickel bromide dimer, methylallylnickel iodide dimer, and ethylallylnickel chloride dimer.

In one embodiment, the organic complex of nickel comprises bis(triphenylphosphine)dicarbonylnickel, methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentylallylnickel chloride, cyclooctenylnickel chloride, cinnamylnickel bromide, phenylallylnickel chloride, cyclohexenylnickel bromide, cyclodecenylnickel chloride, or a combination thereof.

The alkyl aluminum compounds can be alkyl aluminum halides, trialkylaluminum compounds, and/or alkylalumoxanes. The alkyl aluminum halides are primarily the compounds R′AlX₂, R′₂AlX, and mixtures thereof including the mixtures of the formula R′₃Al₂X₃ usually referred to as the sesquihalides. Each R′ can have from 1 to 8 carbon atoms and can be, for example, a methyl, ethyl, propyl, iso-propyl, n-butyl, sec-butyl, tert-butyl, pentyl or hexyl group. Each X can be a fluorine, chlorine, bromine and/or iodine. Examples of suitable alkyl aluminum halides include diethylaluminum chloride, propylaluminum dibromide, dihexylaluminum bromide, ethylaluminum dichloride, n-butylaluminum dibromide, ethyl aluminum sesquichloride, methyl aluminum sesquichloride, ethyl aluminum sesquibromide, and ethyl aluminum sesquifluoride. In one example the alkyl aluminum halides are the chloride and bromide compounds. In another example the alkyl aluminum halides are the chloride compounds.

The trialkylaluminum compounds have the formula R″₃Al. R″ can have from 1 to 8 carbon atoms and can be, for example, a methyl, ethyl, propyl, iso-propyl, n-butyl, sec-butyl, tert-butyl, pentyl or hexyl group. Some examples of suitable trialkylaluminum compounds include trimethylaluminum, triethylaluminum, tri-n-propylaluminum, tri-isopropylaluminum, tri-n-butylaluminum, tri-isobutylaluminum, tri-n-pentylaluminum, tri-n-hexylaluminum, and tri-cyclohexylaluminum.

Some examples of the alkylalumoxanes include methylalumoxane (MAO), polymeric MAO (PMAO), ethylalumoxane, and isobutylalumoxane. For example, the alkylalumoxane can be methylalumoxane.

In one embodiment, the alkyl aluminum compound of the catalyst solution comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

The boranes provide an alternative to the alkyl aluminum compounds since they can act as a Lewis acid in the formation of the catalyst solution. The boranes comprise haloboranes, for example, trifluoroborane, and triarylboranes bearing alkyl, aryl, alkoxy, aryloxy, halide, and haloalkyl substituents. Some additional specific examples of boranes that can be used include, but not limited to, tris(pentafluoro-phenyl)borane, tris(3,5-bis(trifluoromethyl)phenyl)borane, triphenylborane, and mixtures thereof.

In general, the catalyst is prepared in an inert solvent. Examples of solvents include, but are not limited to, alkanes, cycloalkanes, alkenes, cycloalkenes, carbocyclic aromatic compounds, aromatic compounds, esters, ketones, acetals, ethers, halogenated aromatic hydrocarbons, or a mixture thereof. Specific examples of such solvents include alkane and cycloalkanes such as dodecane, decalin, octane, hexane, heptane, iso-octane mixtures, cyclohexane, cyclooctane, cyclododecane, methylcyclohexane or mixtures therof; ethers such as diethyl ether, dipropyl ether, dibutyl ether, tetrahydrofuran; aromatic hydrocarbons such as benzene, toluene, xylene isomers, tetralin, cumene or mixtures therof; alkyl-substituted aromatic compounds such as the isomers of diisopropylbenzene, triisopropylbenzene and tert-butylbenzene, or mixtures therof; crude hydrocarbon mixtures such as naphtha, mineral oils and kerosene or mixtures therof; halogentated aromatic hydrocarbons such as chlorobenzene, 1,2-dichlorobenzene, 1,3-dichlorobenzene, or mixtures thereof.

In one embodiment, the catalyst solution solvent comprises alkanes, cycloalkanes, aromatic hydrocarbons, halogenated aromatic hydrocarbons, ethers, or mixtures thereof. In an additional embodiment of the catalyst solution, the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The individual ligands attached to the phosphorus atom in the tertiary phosphine compounds are t-butyl, 2-pyridyl, 2,6-dimethoxyphenyl, o-tolyl, cyclohexyl, phenyl, butyl, adamantly, 2-pyridyl, 2,6-dimethoxyphenyl, o-tolyl, 2′,4′,6′-triisopropylbiphenyl, 2′-(N,N-dimethylamino)biphenyl, 1-(2,4,6-trimethyl-phenyl)-1H-imidazole, and 1,2,3,4,5-pentaphenyl-1′-ferrocene. These ligands could be used in various combinations or mixtures with phosphorus. The phosphine compounds have the formula: PR¹R²R³ wherein R¹ and R² each are independently selected from the group consisting of t-butyl, 2-pyridyl, 2,6-dimethoxyphenyl, o-tolyl, cyclohexyl, phenyl, butyl, and adamantyl; and wherein R³ is selected from the group consisting of 2-pyridyl, 2,6-dimethoxyphenyl, o-tolyl, 2′,4′,6′-triisopropylbiphenyl, 2′-(N,N-dimethylamino)biphenyl, adamantyl, 1-(2,4,6-trimethyl-phenyl)-1H-imidazole, and 1,2,3,4,5-pentaphenyl-1′-ferrocene.

In one embodiment, the imidazolium or phosphine compounds used with the organic complex of nickel in the catalyst solution comprises triphenylphosphine (I), tri(o-tolyl)phosphine (II), tris(2,6-dimethoxyphenyl)phosphine (III), tri-2-pyridylphosphine (IV), tri-tert-butylphosphine (V), 2-dicyclohexylphosphino-2′,4′,6′-triisopropylbiphenyl (VI), 2-di-tert-butylphosphino-2′,4′,6′-triisopropylbiphenyl (VII), 2-diphenylphosphino-2′-(N,N-dimethylamino)biphenyl (VIII), 2-(dicyclohexylphosphino)-1-(2,4,6-trimethyl-phenyl)-1H-imidazole (IX), di(1-adamantyl)-n-butylphosphine (X), di(1-adamantyl)benzylphosphine (XI), 1,3-bis(2,4,6-trimethylphenyl)-1,3-dihydro-2H-imidazol-2-ylidene (XII), and 1,2,3,4,5-pentaphenyl-1′-(di-t-butylphosphino)ferrocene (XIII). The corresponding chemical structures for these thirteen compounds or ligands are given here:

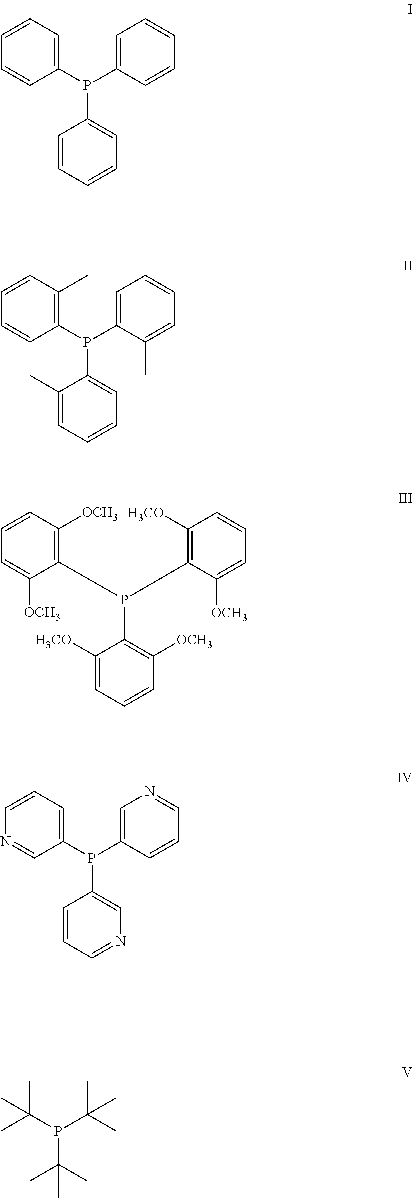 [SMILES: c1ccc(P(c2ccccc2)c2ccccc2)cc1 ; Cc1ccccc1P(c1ccccc1C)c1ccccc1C ; COc1cccc(OC)c1P(c1c(OC)cccc1OC)c1c(OC)cccc1OC ; c1cncc(P(c2cccnc2)c2cccnc2)c1 ; CC(C)(C)P(C(C)(C)C)C(C)(C)C]

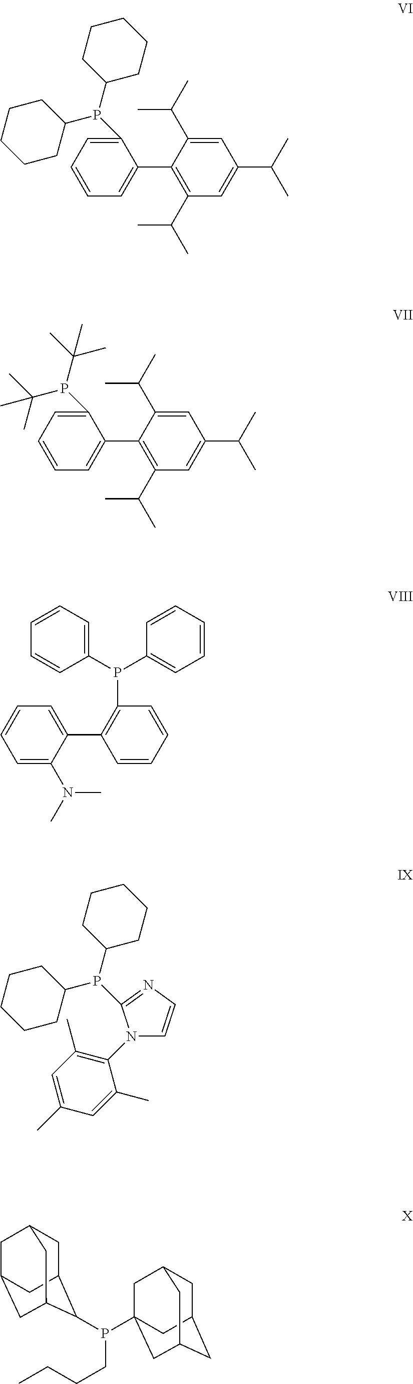

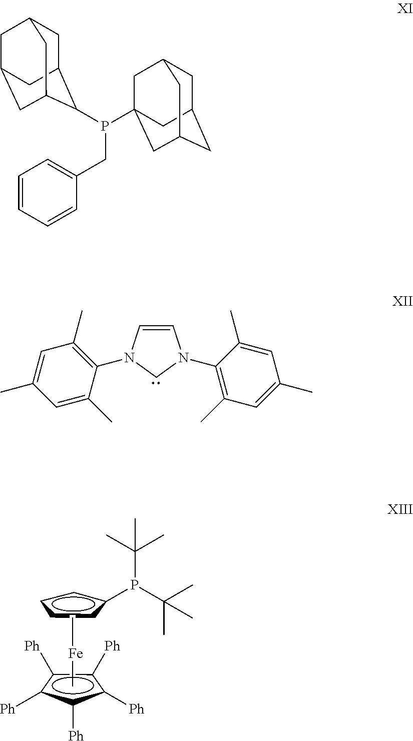

The concentration of nickel (denoted as ‘[Ni]’) ranges from about 1 mmol/L to about 100 mmol/L. Some additional examples of nickel concentration include from about 1 mmol/L to about 25 mmol/L, and about 1 mmol/L to about 5 mmol/L.

The molar ratio of alkyl aluminum compound to the organic complex of nickel is about 100,000 to about 1. In another embodiment the molar ratio of alkyl aluminum compound to the organic complex of nickel is about 10,000 to about 1, in another embodiment about 100 to about 1, and in another embodiment the molar ratio of alkyl aluminum compound to nickel is about 20 to about 1.

The molar ratio of phosphine compound to the organic complex of nickel is about 0.1 to about 2. In another embodiment the molar ratio of phosphine compound to the organic complex of nickel is about 1 to about 2, and in another embodiment the molar ratio of phosphine ligand to nickel is about 1.5 to about 2. Molar ratios of phosphine compound to organic nickel complex greater than 2 will begin to attenuate catalyst activity.

The alkyl aluminum compound may be first added to the organic complex of nickel followed by the addition of the phosphine compound; in one example, all of the additions are performed in the presence of a solvent. In some instances reversing the addition of the alkyl aluminum compound with the phosphine compound may lead to a less active catalyst solution.

In a second embodiment, the present invention is a catalyst solution comprising: (i) an allylnickel halide catalyst; (ii) an alkyl aluminum compound; (iii) a solvent; and (iv) at least one phosphine compound selected from the following structures:

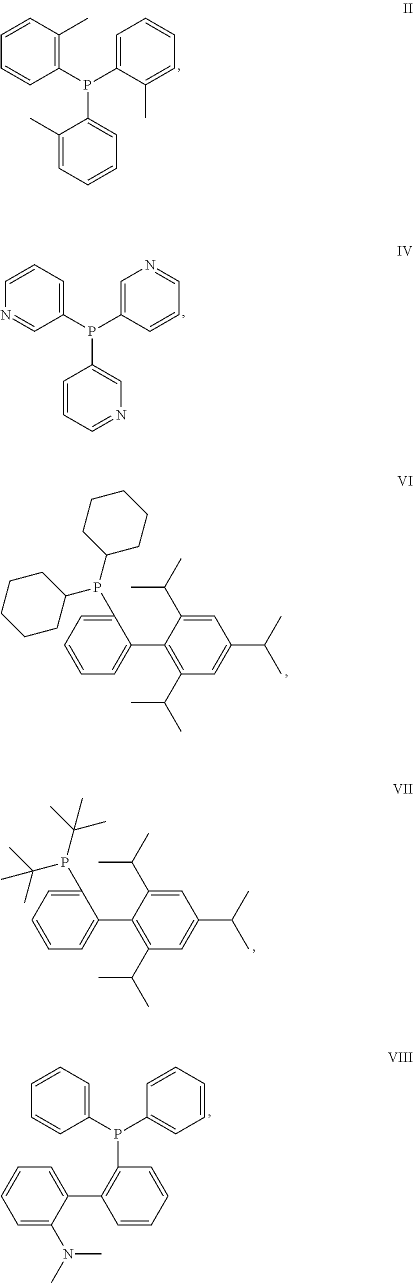

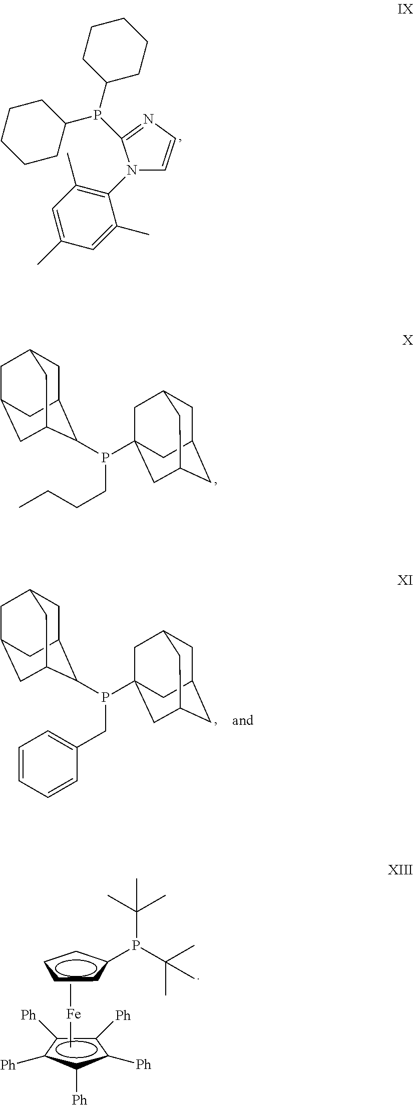

It is understood that the description of the catalyst solution previously discussed, which can be used in any combination, apply equally well to the second embodiment of the catalyst solution.

In one embodiment, the allylnickel halide of the catalyst solution comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentylallylnickel chloride, cyclooctenylnickel chloride, cinnamylnickel bromide, phenylallylnickel chloride, cyclohexenylnickel bromide, cyclodecenylnickel chloride, or combinations thereof.

In one embodiment, the alkyl aluminum compound of the catalyst solution comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

In one embodiment, the catalyst solution solvent comprises alkanes, cycloalkanes, aromatic hydrocarbons, halogenated aromatic hydrocarbons, ethers, or mixtures thereof. In an additional embodiment of the catalyst solution, the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The phosphine compound used with the allylnickel halide catalyst in the catalyst solution consists of tri(o-tolyl)phosphine (II), tri-2-pyridylphosphine (IV), 2-dicyclohexylphosphino-2′,4′,6′-triisopropylbiphenyl (VI), 2-di-tert-butylphosphino-2′,4′,6′-triisopropylbiphenyl (VII), 2-diphenylphosphino-2′-(N,N-dimethylamino)biphenyl (VIII), 2-(dicyclohexylphosphino)-1-(2,4,6-trimethyl-phenyl)-1H-imidazole (IX), di(1-adamantyl)-n-butylphosphine (X), di(1-adamantyl)benzylphosphine (XI), and 1,2,3,4,5-pentaphenyl-1′-(di-t-butylphosphino)ferrocene (XIII). The corresponding chemical structures for these compounds or ligands are given here:

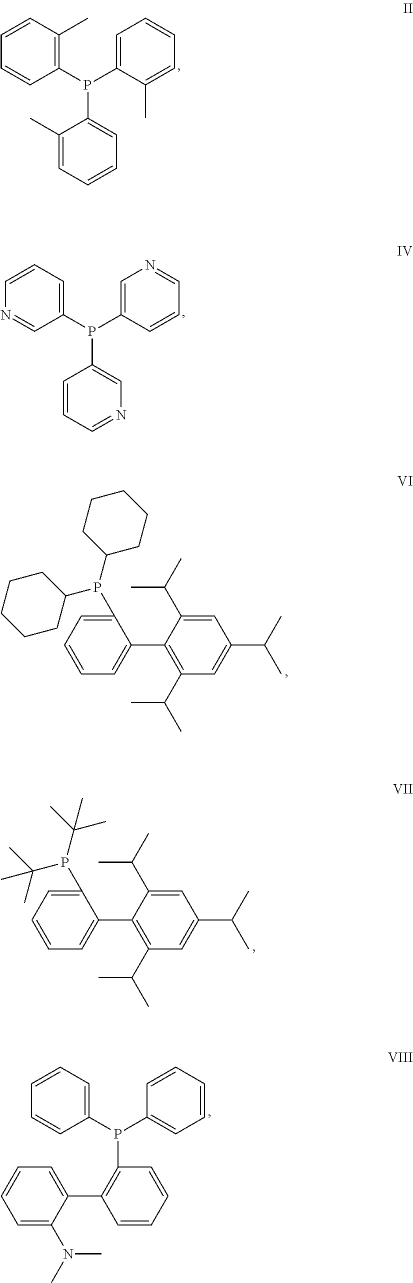

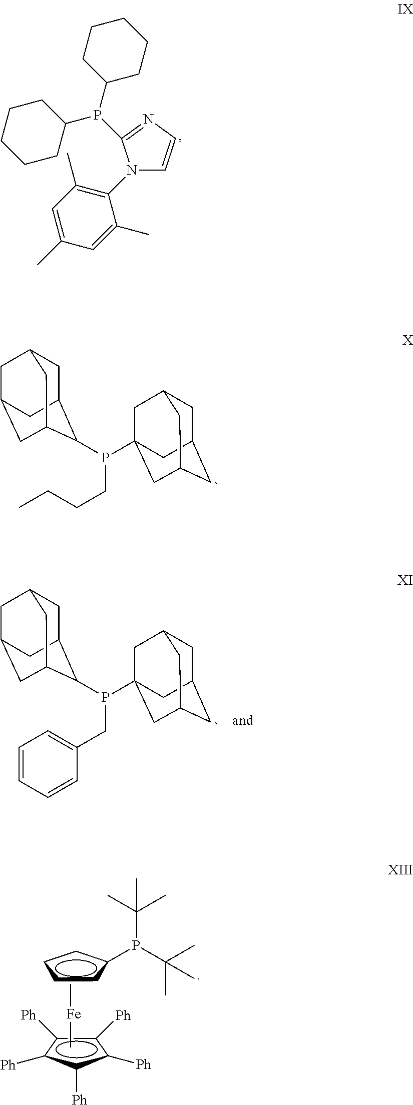

In one embodiment, the phosphine compound used with the allylnickel halide catalyst in the catalyst solution consists of 2-dicyclohexylphosphino-2′,4′,6′-triisopropylbiphenyl (VI), 2-di-tert-butylphosphino-2′,4′,6′-triisopropylbiphenyl (VII), di(1-adamantyl)-n-butylphosphine (X), di(1-adamantyl)benzylphosphine (XI), and 1,2,3,4,5-pentaphenyl-1′-(di-t-butylphosphino)ferrocene (XIII). The corresponding chemical structures for these compounds are given here:

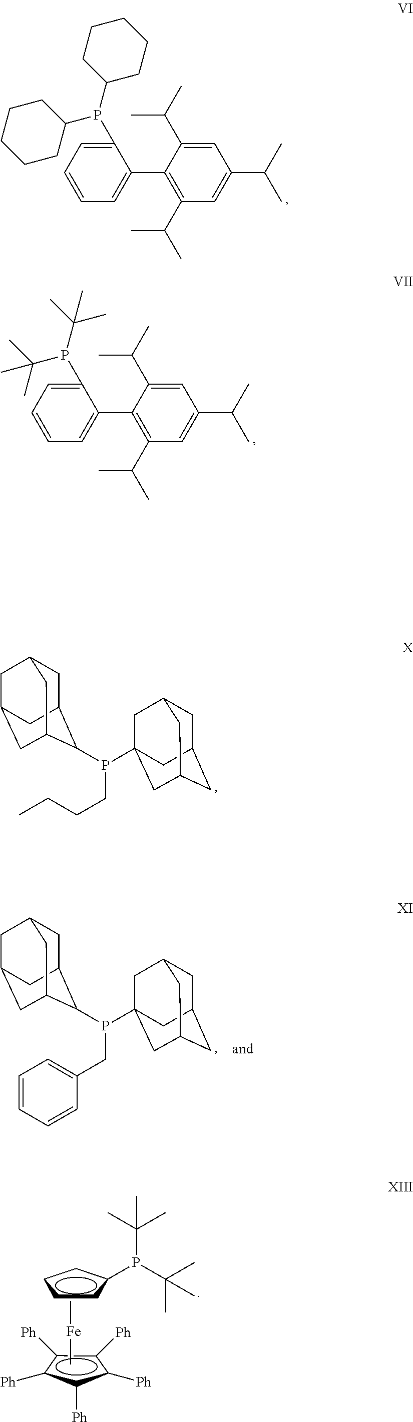

In another embodiment, the phosphine compound used with the allylnickel halide catalyst in the catalyst solution consist of 2-dicyclohexylphosphino-2′,4′,6′-triisopropylbiphenyl (VI), and 2-di-tert-butyl phosphino-2′,4′,6′-triisopropylbiphenyl (VII). The corresponding chemical structures for these two compounds are given here:

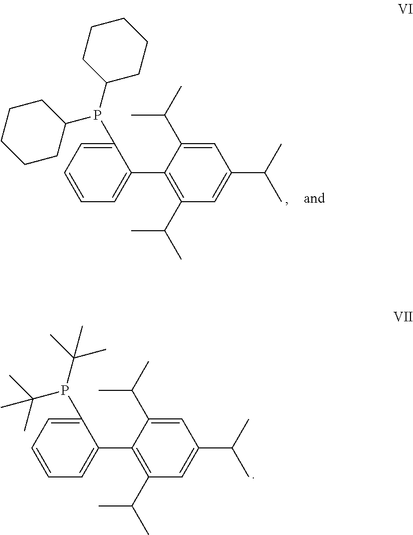

In a third embodiment, the present invention is an ethylene and propylene co-dimerization process comprising: contacting ethylene and propylene under elevated pressure with a catalyst solution comprising: (i) an allylnickel halide catalyst; (ii) an alkyl aluminum compound; (iii) a solvent; and (iv) at least one phosphine compound selected from the following structures:

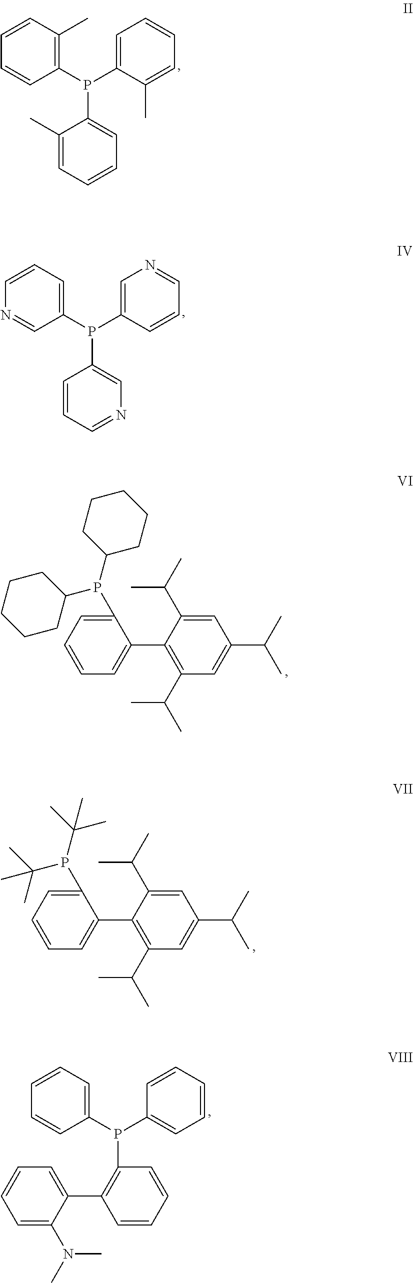

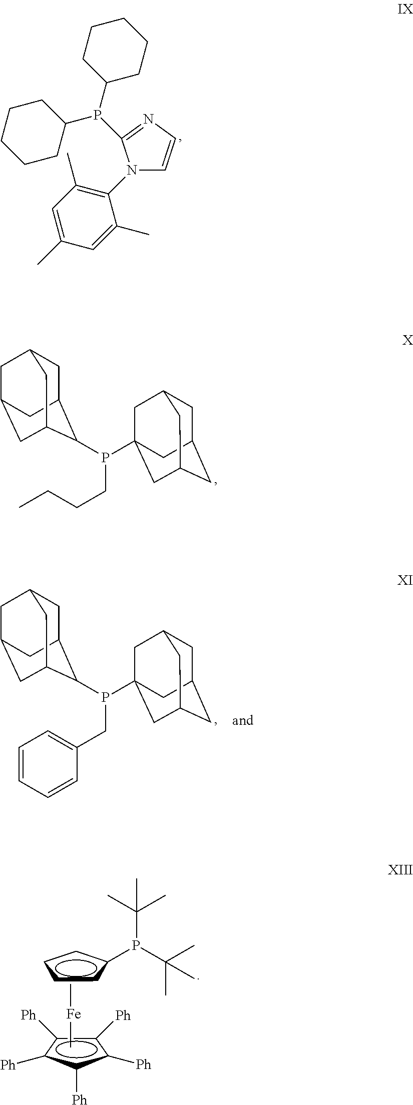

It is understood that the descriptions of the catalyst solution previously discussed, which can be used in any combination, apply equally well to the third embodiment of the catalyst solution used for the ethylene and propylene co-dimerization process.

In one embodiment, the allylnickel halide of the catalyst solution comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentylallylnickel chloride, cyclooctenylnickel chloride, cinnamylnickel bromide, phenylallylnickel chloride, cyclohexenylnickel bromide, cyclodecenylnickel chloride, or a combination thereof.

In one embodiment, the alkyl aluminum compound of the catalyst solution comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

In one embodiment, the catalyst solution solvent comprises alkanes, cycloalkanes, aromatic hydrocarbons, halogenated aromatic hydrocarbons, ethers, or mixtures thereof. In an additional embodiment of the catalyst solution, the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The molar ratio of ethylene to propylene, fed as reactive gases, can be up to about 1:100, about 1:25, about 1:10, or about 1:2, all with excess propylene. The molar ratio of ethylene to propylene, switched now for excess ethylene, can be up to about 100:1, about 25:1, about 10:1, or about 2:1. In one embodiment, the molar ratio of ethylene:propylene is about 100:1 to about 1:100. In another embodiment, the molar ratio of ethylene:propylene is about 10:1 to about 1:10. With excess ethylene the C4 and C5 products would be preferred since both the reactant stoichiometry and catalyst solution reactivity would both favor product compositions that favored C4's and C5's. Even when the feedstock contains an excess of propylene, the catalyst solutions of this invention have the ability to minimize the amount of C6 olefins and maximize both C4s and C5s produced. About a 1:1 or equal molar ratio of ethylene to propylene can also be used.

The operating temperature for this co-dimerization process can range from about −80° C. to about 100° C. In one example, the operating temperature for this co-dimerization process can range from about 0° C. to about 50° C. In one embodiment, the ethylene and propylene are contacted with the catalyst solution at a temperature of about −80° C. to about 100° C.

This co-dimerization process can be run at a pressure from about 1 atm to about 70 atm. In another embodiment this co-dimerization process can be run at a pressure from about 1 atm to about 25 atm and in another embodiment about 1.5 atm to about 7 atm. In one embodiment, the ethylene and propylene are added to the catalyst solution wherein the contacting is at a pressure of about 1.5 atm to about 7 atm.

The co-dimerization reaction may be carried out in a variety of reactor types including, but not limited to, stirred tank, continuous stirred tank, and tubular reactors. Any of the known olefin oligomerization reactor designs or configurations may be used for the co-dimerization reaction to produce the olefin product. For example, the process may be conducted in a batchwise manner in an autoclave by contacting the ethylene and propylene in the presence of the catalyst compositions described herein. It will be apparent to those skilled in the art that other reactor schemes may be used with this invention. For example, the co-dimerization reaction can be conducted in a plurality of reaction zones, in series, in parallel, or it may be conducted batchwise or continuously in a tubular plug flow reaction zone or series of such zones with recycle of unconsumed feed olefin substrate materials if required. The reaction steps may be carried out by the incremental addition of one of the feed olefin substrate materials to the other. Also, the reaction steps can be combined by the joint addition of the feed olefin substrate materials.

After the dimerization reaction, the reaction is terminated by depressurizing or venting the reactor or by deactivating the catalyst with a reactive alcohol containing molecules such as water, methanol, ethanol or propanol. In a batch process, the reaction time may be determined by the liquid volume of the reactor, which increases as olefin reactants and products dissolve in the solvent. In continuous operation mode the reaction time may be controlled by the residence time within the reactor. The residence time is determined by the liquid velocity through the reactor and is also dependent on reactor temperature and pressure.

It is to be understood that the mention of one or more process steps does not preclude the presence of additional process steps before or after the combined recited steps or intervening process steps between those steps expressly identified. Moreover, the lettering of process steps or ingredients is a convenient means for identifying discrete activities or ingredients and the recited lettering can be arranged in any sequence, unless otherwise indicated.

### LIST OF NON-LIMITING EMBODIMENTS

Embodiment A is a catalyst solution comprising: (i) an organic complex of nickel; (ii) an alkyl aluminum compound; (iii) a solvent; and (iv) at least one phosphine compound having the formula: PR¹R²R³ wherein R¹ and R² each are independently selected from the group consisting of t-butyl, 2-pyridyl, 2,6-dimethoxyphenyl, o-tolyl, cyclohexyl, phenyl, butyl, and adamantyl; and wherein R³ is selected from the group consisting of 2-pyridyl, 2,6-dimethoxyphenyl, o-tolyl, 2′,4′,6′-triisopropylbiphenyl, 2′-(N,N-dimethylamino)biphenyl, adamantyl, 1-(2,4,6-trimethyl-phenyl)-1H-imidazole, and 1,2,3,4,5-pentaphenyl-1′-ferrocene.

The catalyst solution of Embodiment A wherein the organic complex of nickel comprises bis(triphenylphosphine)dicarbonylnickel, methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentyallylnickel chloride, cyclooctenylnickel chloride, cinnamylnickel bromide, phenylallylnickel chloride, cyclohexenylnickel bromide, cyclodecenylnickel chloride, or a combination thereof.

The catalyst solution of Embodiment A or Embodiment A with one or more of the intervening features which further comprises a solvent wherein the solvent comprises alkanes, cycloalkanes, aromatic hydrocarbons, halogenated aromatic hydrocarbons, ethers, or mixtures thereof.

The catalyst solution of Embodiment A or Embodiment A with one or more of the intervening features which further comprises a solvent comprising alkanes, cycloalkanes, aromatic hydrocarbons, halogenated aromatic hydrocarbons, ethers, or mixtures thereof wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The catalyst solution of Embodiment A or Embodiment A with one or more of the intervening features which further comprises an alkyl aluminum compound wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

Embodiment B is a catalyst solution comprising: (i) an allylnickel halide catalyst; (ii) an alkyl aluminum compound; (iii) a solvent; and (iv) at least one phosphine compound selected from the following structures:

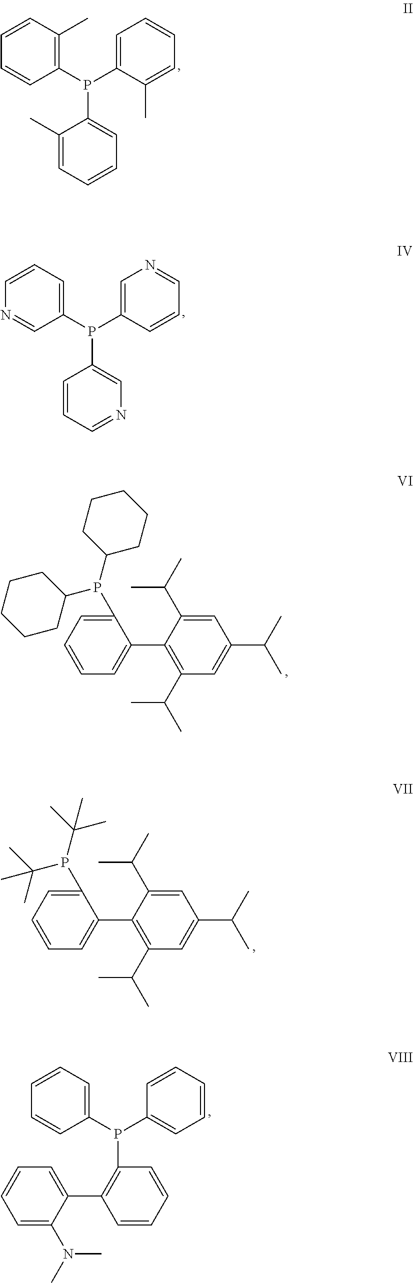

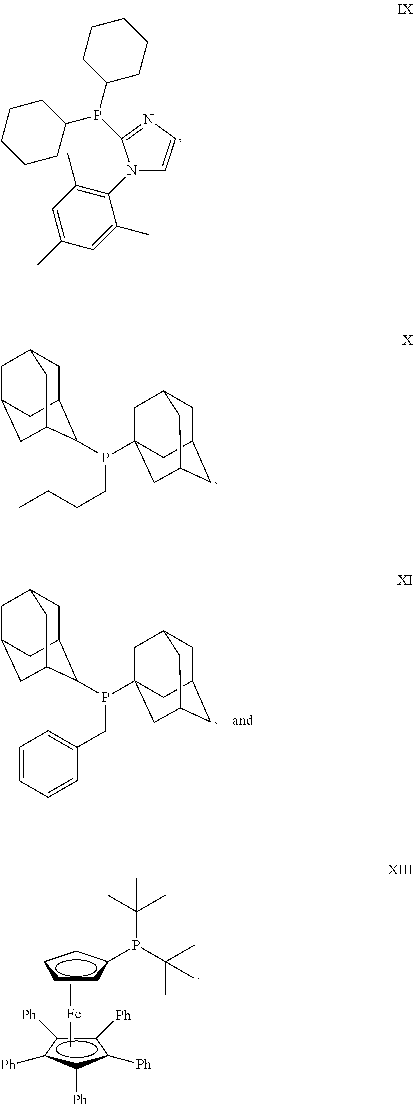

The catalyst solution of Embodiment B wherein the allylnickel halide comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentyallylnickel chloride, cyclooctenylnickel chloride, cinnamylnickel bromide, phenylallylnickel chloride, cyclohexenylnickel bromide, cyclodecenylnickel chloride, or combinations thereof.

The catalyst solution of Embodiment B or Embodiment B with one or more of the intervening features which further comprises a solvent wherein the solvent comprises alkanes, cycloalkanes, aromatic hydrocarbons, halogenated aromatic hydrocarbons, ethers, or mixtures thereof.

The catalyst solution of Embodiment B or Embodiment B with one or more of the intervening features which further comprises a solvent comprising alkanes, cycloalkanes, aromatic hydrocarbons, halogenated aromatic hydrocarbons, ethers, or mixtures thereof wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The catalyst solution of Embodiment B or Embodiment B with one or more of the intervening features which further comprises an alkyl aluminum compound wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

Embodiment C is an ethylene and propylene co-dimerization process comprising contacting ethylene and propylene under elevated pressure with a catalyst solution comprising: (i) an allylnickel halide catalyst; (ii) an alkyl aluminum compound; (iii) a solvent; and (iv) at least one phosphine compound selected from the following structures:

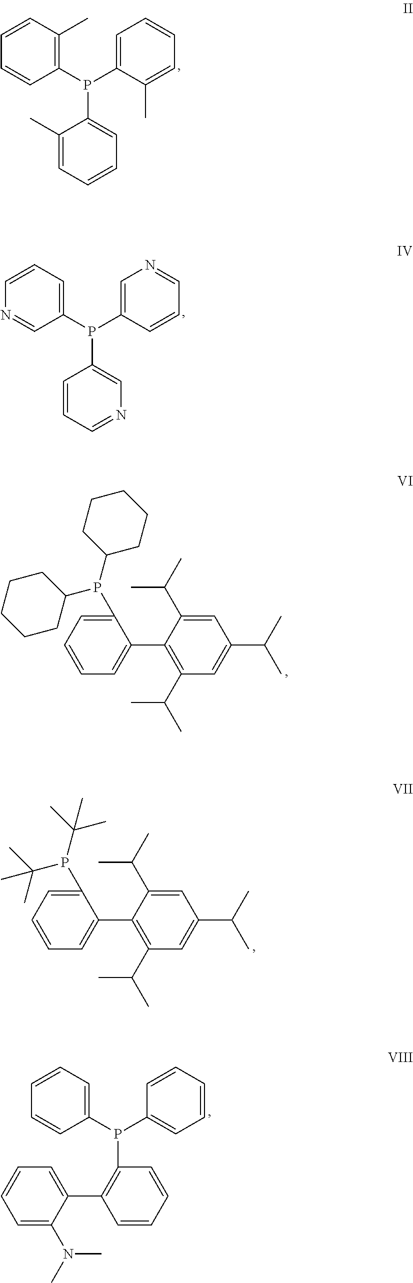

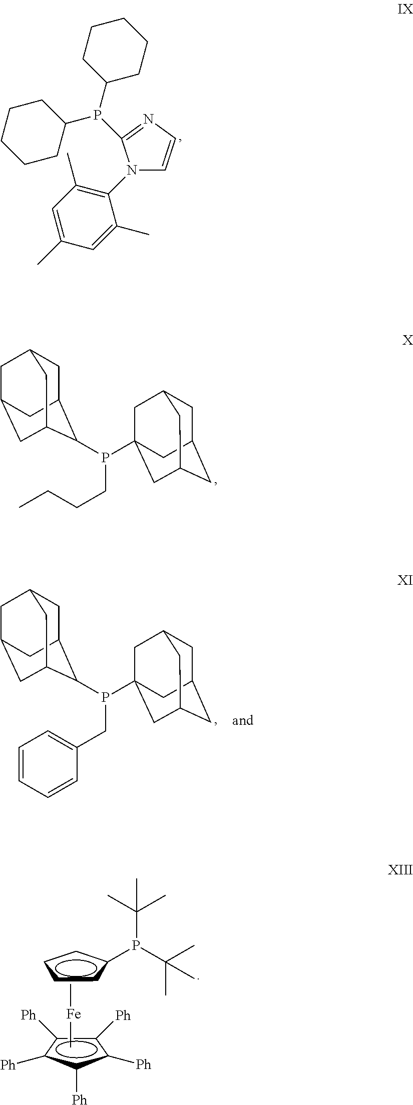

The process of Embodiment C wherein the allylnickel halide catalyst comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentyallylnickel chloride, cyclooctenylnickel chloride, cinnamylnickel bromide, phenylallylnickel chloride, cyclohexenylnickel bromide, cyclodecenylnickel chloride, or a combination thereof.

The process of Embodiment C or Embodiment C with one or more of the intervening features which further comprises a solvent wherein the solvent comprises alkanes, cycloalkanes, aromatic hydrocarbons, halogenated aromatic hydrocarbons, ethers, or mixtures thereof.

The process of Embodiment C or Embodiment C with one or more of the intervening features which further comprises a solvent comprising alkanes, cycloalkanes, aromatic hydrocarbons, halogenated aromatic hydrocarbons, ethers, or mixtures thereof wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

The process of Embodiment C or Embodiment C with one or more of the intervening features which further comprises an alkyl aluminum compound wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

The process of Embodiment C or Embodiment C with one or more of the intervening features which further comprises a molar ratio of ethylene:propylene wherein the molar ratio of ethylene:propylene is about 100:1 to about 1:100.

The process of Embodiment C or Embodiment C with one or more of the intervening features which further comprises a molar ratio of ethylene:propylene wherein the molar ratio of ethylene:propylene is about 10:1 to about 1:10.

The process of Embodiment C or Embodiment C with one or more of the intervening features which further comprises temperature conditions wherein the ethylene and propylene are contacted with the catalyst solution at a temperature of about −80 to about 100° C.

The process of Embodiment C or Embodiment C with one or more of the intervening features which further comprises contacting pressure conditions wherein the ethylene and propylene are added to the catalyst solution wherein the contacting is at a pressure of about 1.5 to about 7 atm.

The process of Embodiment C or Embodiment C with one or more of the intervening features which further comprises molar ratios of components wherein the molar ratio of alkyl aluminum compound to nickel is about 1:20; and wherein the molar ratio of phosphine compound to nickel is about 1.5:2.

### EXAMPLES

### General

The nickel precursors, solvents, ethylene, propylene, and ethylaluminum sesquichloride were purchased from commercial suppliers. The phosphine compounds or ligands: triphenylphosphine (I), tri(o-tolyl)phosphine (II), tris(2,6-dimethoxyphenyl)phosphine (III), tri-2-pyridylphosphine (IV), tri-tert-butylphosphine (V), 2-dicyclohexylphosphino-2′,4′,6′-triisopropyl biphenyl (VI), 2-di-tert-butylphosphino-2′,4′,6′-triisopropyl biphenyl (VII), 2-diphenylphosphino-2′-(N, N-dimethylamino)biphenyl (VIII), 2-(dicyclohexylphosphino)-1-(2,4,6-trimethyl-phenyl)-1H-imidazole (IX), di(1-adamantyl)-n-butylphosphine (X), di(1-adamantyl)benzylphosphine (XI), 1,3-bis(2,4,6-trimethylphenyl)-1,3-dihydro-2H-imidazol-2-ylidene (XII), and 1,2,3,4,5-pentaphenyl-1′-(di-t-butylphosphino)ferrocene (XIII) were purchased from commercial suppliers (Aldrich Chemical Co., and Sigma) and used as received.

### ABBREVIATIONS

The following abbreviations are used throughout the Examples: Ni=Nickel, C4=butene isomers, C5=pentene isomers, C6=hexene isomers, LA=Lewis Acid, atm=Atmosphere, SCCM=standard cubic centimeters per minute, mmol=millimoles, prod=product, GC=Gas Chromatography.

Gas Chromatography Measurements—

A typical product analysis entailed chilling the product bottle in air to 5° C. for a minimum of one hour. An internal standard solution was prepared by adding 75.0 g of cyclopentanone to a 1 L flask and then filling to volume with acetonitrile. Approximately 1.0 g of product sample was then weighed into a 4 dram vial along with 7.86 g of the internal standard solution. The contents were then mixed and transferred to a GC vial for analysis. Each GC sample was injected on a Shimadzu 2010 gas chromatograph with an AOC-20 autosampler and the components separated by an HP-Al2O3/M column (50 m×0.32 mm×8 μm) and analyzed by FID. Product peaks were identified by comparison to known standards or by mass spectrometric characterization.

Calculations—

The following calculations were used to determine the percentage of C4, C5, and C6 olefin products detected by GC and the relative amounts of linear and branched C5 products. Only isomers detectable through GC are listed for the hexene or C6 calculations although some isomers listed were present in trace amounts.

GC wt. % of Total C4s=% trans-2-butene+% 1-butene+% 2-methyl-propene+% cis-2-butene

GC wt. % of Total C5s=% trans-2-pentene+% 2-methyl-2-butene+% 1-pentene+% 2-methyl-1-butene+% cis-2-pentene

GC wt. % of Total C6s=% trans-4-methyl-2-pentene+% cis-4-methyl-2-pentene+% 2,3-dimethyl-1-butene+% trans-2-hexene+% 2-ethyl-1-butene+% 2-methyl-1-pentene+% cis-3-hexene+% 1-hexene+% cis-2-hexene+% trans-3-hexene+% 2-methyl-2-pentene+% 3-methyl-2-pentene

% Linear C5s in C5 Fraction=(% trans-2-pentene+% 1-pentene+cis-2-pentene)/(GC wt. % of Total C5s)×100%

% Branched C5s in C5 Fraction=(% 2-methyl-2-butene+% 2-methyl-1-butene)/(GC wt. % of Total C5s)×100%

C4 mass fraction=(molar mass of butenes)/(molar mass of butenes+molar mass of pentenes+molar mass of hexenes)

C5 mass fraction=(molar mass of pentenes)/(molar mass of butenes+molar mass of pentenes+molar mass of hexenes)

C6 mass fraction=(molar mass of hexenes)/(molar mass of butenes+molar mass of pentenes+molar mass of hexenes)

Mole % C4s=[(GC wt. % of Total C4s)/(C4 mass fraction)]/[(GC wt. % of Total C4s)/(C4 mass fraction)+(GC wt. % of Total C5s)/(C5 mass fraction)+(GC wt. % of Total C6s)/(C6 mass fraction)]×100%

Mole % C5s=[(GC wt. % of Total C5s)/(C5 mass fraction)]/[(GC wt. % of Total C4s)/(C4 mass fraction)+(GC wt. % of Total C5s)/(C5 mass fraction)+(GC wt. % of Total C6s)/(C6 mass fraction)]×100%

Mole % C6s=[(GC wt. % of Total C6s)/(C6 mass fraction)]/[(GC wt. % of Total C4s)/(C4 mass fraction)+(GC wt. % of Total C5s)/(C5 mass fraction)+(GC wt. % of Total C6s)/(C6 mass fraction)]×100%

Catalyst Synthesis—

The thermally sensitive methylallylnickel chloride dimer was purified upon receipt from Strem by dissolution in dry, degassed toluene under inert atmosphere followed by filtration through Celite and then removal of the toluene under vacuum. The alkyl aluminum halide, ethylaluminum sesquichloride, was diluted to 37 percent by weight with dry, degassed toluene to form a stock solution.

Catalyst syntheses were carried out in an inert atmosphere drybox using anhydrous degassed solvents. In preparation of a co-dimerization experiment, the catalyst solution was prepared by adding approximately 20 mg of either bis(triphenylphosphine)dicarbonylnickel or methylallylnickel chloride precursor to a 50 mL Schlenk flask followed by dissolution in about 10 mL of toluene. Next, 0.4 g of the 37 wt. % ethylaluminum sesquichloride stock solution was diluted with an additional 10 mL of toluene and then added to the Schlenk flask to give an orange-red solution. In the experiments using a phosphine or additional ligand, the selected ligand was dissolved in about 10 mL of toluene and then added to the Schlenk flask with the nickel solution. The thirteen compounds or ligands used in these experiments are shown below and are labeled I-XIII.

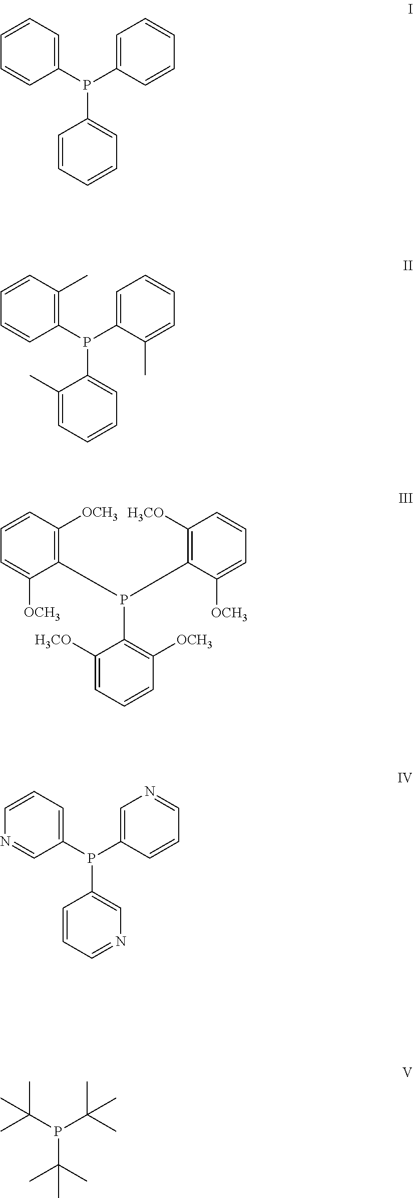 [SMILES: c1ccc(P(c2ccccc2)c2ccccc2)cc1 ; Cc1ccccc1P(c1ccccc1C)c1ccccc1C ; COc1cccc(OC)c1P(c1c(OC)cccc1OC)c1c(OC)cccc1OC ; c1cncc(P(c2cccnc2)c2cccnc2)c1 ; CC(C)(C)P(C(C)(C)C)C(C)(C)C]

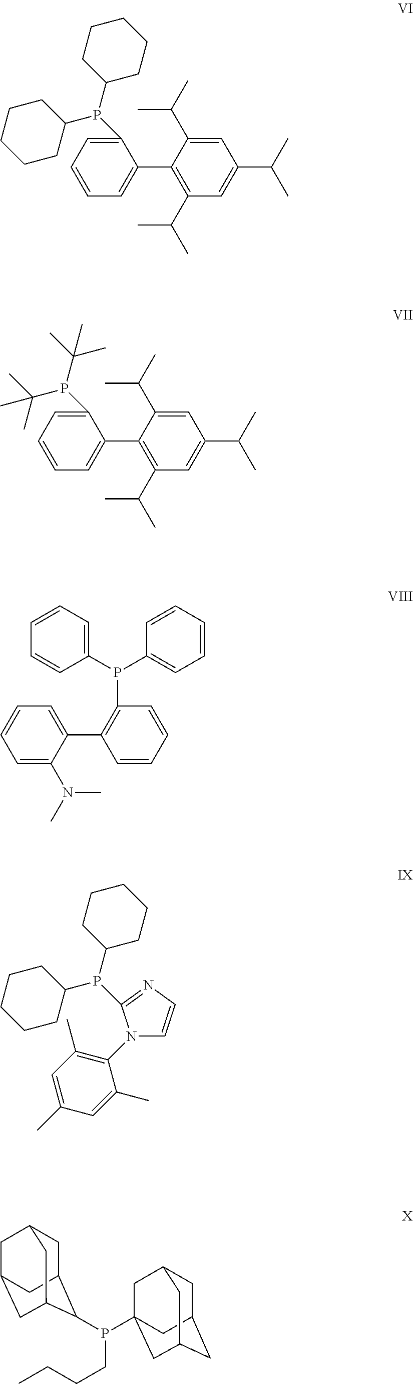

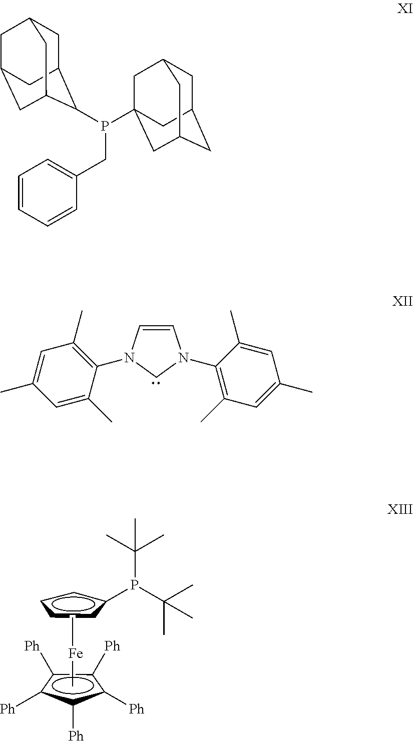

### Examples 1-28

The synthesis of twenty eight different catalyst solutions were prepared according to the methods described above with the various imidazolium and phosphine ligands. The concentration of the organic complex of nickel (denoted as ‘[Ni]’) ranged from about 2 to 11 mmol/L. In Examples 1-3 the nickel was introduced from nickel dicarbonyl bis(triphenylphosphine) (denoted as Ni(CO)₂(PPh₃)₂ in Table 1) while in Examples 4-28 the nickel was introduced from methylallylnickel chloride (denoted as CH₃(allyl)NiCl₂ in Table 1). The ratio of alkyl aluminum compound (methylaluminum sesquichloride) to nickel precursor (denoted as ‘Al/Ni’) ranged from about 2 to 11 while the ratio of phosphine ligand to nickel (denoted as ‘L/Ni’) ranged from about 1.5 to 2.1. Table 1 lists the catalyst solutions prepared and the relative concentrations and/or stoichiometries of the different components.

TABLE 1
Prepared Catalyst Solutions

| Example | Ni Source | [Ni] (mmol/L) | Al/Ni | Ligand | L/Ni |
|---|---|---|---|---|---|
| 1 | Ni(CO)₂(PPh₃)₂ | 10.7 | 2.1 | none | — |
| 2 | Ni(CO)₂(PPh₃)₂ | 10.7 | 2.1 | VII | 0.4 |
| 3 | Ni(CO)₂(PPh₃)₂ | 10.9 | 2.1 | X | 0.4 |
| 4 | CH₃(allyl)NiCl₂ | 2.2 | 10.3 | none | — |
| 5 | CH₃(allyl)NiCl₂ | 2.2 | 10.2 | VI | 1.9 |
| 6 | CH₃(allyl)NiCl₂ | 2.2 | 10.1 | X | 2.0 |
| 7 | CH₃(allyl)NiCl₂ | 2.7 | 8.6 | none | — |
| 8 | CH₃(allyl)NiCl₂ | 2.6 | 8.9 | VII | 1.8 |
| 9 | CH₃(allyl)NiCl₂ | 2.2 | 10.2 | none | — |
| 10 | CH₃(allyl)NiCl₂ | 2.1 | 10.8 | VII | 2.0 |
| 11 | CH₃(allyl)NiCl₂ | 2.7 | 8.4 | IX | 1.6 |
| 12 | CH₃(allyl)NiCl₂ | 2.6 | 8.9 | XI | 1.7 |
| 13 | CH₃(allyl)NiCl₂ | 2.8 | 9.5 | XIII | 1.7 |
| 14 | CH₃(allyl)NiCl₂ | 2.5 | 8.9 | III | 1.7 |
| 15 | CH₃(allyl)NiCl₂ | 2.6 | 9 | XII | 1.7 |
| 16 | CH₃(allyl)NiCl₂ | 2.3 | 9.6 | none | — |
| 17 | CH₃(allyl)NiCl₂ | 2.3 | 9.7 | VII | 2.0 |
| 18 | CH₃(allyl)NiCl₂ | 2.2 | 10.1 | none | — |
| 19 | CH₃(allyl)NiCl₂ | 2.2 | 10.1 | VI | 2.0 |
| 20 | CH₃(allyl)NiCl₂ | 2.7 | 8.5 | X | 1.6 |
| 21 | CH₃(allyl)NiCl₂ | 2.2 | 10 | V | 1.6 |
| 22 | CH₃(allyl)NiCl₂ | 2.2 | 10.2 | I | 2.0 |
| 23 | CH₃(allyl)NiCl₂ | 2.2 | 10.1 | IV | 1.9 |
| 24 | CH₃(allyl)NiCl₂ | 2.2 | 10 | VIII | 2.0 |
| 25 | CH₃(allyl)NiCl₂ | 2.1 | 10.7 | II | 2.1 |
| 26 | CH₃(allyl)NiCl₂ | 3 | 7.8 | none | — |
| 27 | CH₃(allyl)NiCl₂ | 2.6 | 8.6 | VII | 1.7 |
| 28 | CH₃(allyl)NiCl₂ | 2.3 | 9.6 | VI | 1.9 |

Co-Dimerization—

A lab-scale 75 mL Fisher-Porter reactor equipped with a gas inlet line and separate sampling line was used for the co-dimerization experiments with pressurized ethylene and propylene. Once the catalyst solution was prepared the Schlenk flask was capped with a pierced septum and removed from the drybox. The catalyst solution was charged to the Fisher-Porter reactor via the sampling line with the aid of argon pressure. The reactor was then pressurized with a mixture of ethylene and propylene and this initial pressure was maintained for the duration of the experiment using the ethylene/propylene mixture. All co-dimerization reactions were performed at either room temperature (‘RT’), 0° C. by immersing the reactor in an ice-water bath, or 40° C. by using a heated oil bath. Upon completion of the reaction, the reactor was vented and the product solution analyzed by GC.

Calculated Statistical Distribution of Products—

The following examples are calculations that illustrate the statistical distribution of products that would be expected from simple statistical calculations for the co-dimerization of ethylene and propylene in a 1:1, 1:2, or 1:9 ratio. These mixtures of reactant gases allow for ethylene to react with another ethylene to form butene or to react with propylene to form both branched and linear pentenes. The propylene gas reagent can either react with another propylene molecule to form both linear and branched hexenes or the propylene reagent can react with ethylene to form both linear and branched pentenes. The variety of products that can be formed depends on the reactivity of the co-dimerization catalyst towards ethylene and propylene. Product distribution can also depend on the relative concentrations of the ethylene and propylene reagent gases. The greater molar concentration of either ethylene or propylene with respect to the other would normally produce a greater yield of butene or hexene products, respectively. Depending on initial reagent gas concentrations and assuming the relative reactivity of ethylene and propylene with the co-dimerization catalyst are equal, Table 2 displays the calculated product distribution of C4s, Cys, and C6s.

TABLE 2
Calculated Distribution of Products

| | | C4% of Final | C5% of Final | C6% of Final | Product Ratio |
|---|---|---|---|---|---|
| Example | Ethylene:Propylene | Product | Product | Product | C4's:C5's:C6's |
| Calculated Distribution 1 | 1:1 | 0.25 | 0.50 | 0.25 | 1:2:1 |
| Calculated Distribution 2 | 1:2 | 0.11 | 0.44 | 0.44 | 1:4:4 |
| Calculated Distribution 3 | 1:9 | 0.01 | 0.18 | 0.81 | 1:18:81 |

Table 3 displays the co-dimerization results of the twenty eight catalyst solutions presented in Table 1. Examples are formatted so that the catalyst solution from Example 1 is the catalyst used in co-dimerization Example 1A.

### Examples 1A-3A

The effect of sterically hindered phosphines on the zerovalent nickel complex Ni(CO)₂(PPh₃)₂ for the co-dimerization of ethylene and propylene in a 1:2 ethylene:propylene atmosphere at room temperature is examined in Examples 1A-3A shown in Table 3. Example 1A, the control reaction, shows that the C6 olefins make up 57% of the product distribution while the C4's and C5's products make up 24% and 19%, respectively. The reactions in Examples 2A-3A demonstrate that ligand VII increases the mole % of C4s from 24% to 68% and decreases the mole % of C6s from 57% to 12% while ligand X increases the mole % of C4's from 24% to 75% and decreases the mole % of C6 from 57% down to 4%. Irrespective of the phosphine ligand used in these three examples, the distribution of linear and branched pentenes is about equal.

### Examples 4A-6A

The type of phosphine compound used with a methylallylnickel chloride complex can affect both the relative distribution of C4 to C6 olefins and the catalyst selectivity toward linear and branched C5 olefins when a 1:2 ethylene:propylene atmosphere is used at 0° C. (Examples 4A-6A). The product distribution in the control example (Example 4A) produced 48% hexenes, 14% C4's, and 39% C5's. The use of ligand VI (Example 5A) increased the mole % of C4's from 14% in the control to 83% and decreased the mole % of C6 from 48% in the control down to 2%. Ligand X (Example 6A) decreased the mole % of C4's from 14% in the control to 8%, decreased the mole % of C5's from 39% in the control to 38%, and increased the mole % of C6's from 48% to 54%. Ligand X of Example 6A also inverted the linear to branched C5 olefin selectivity relative to the control (Example 4A) with 22% linear pentenes and 78% branched pentenes.

### Examples 7A-8A

The reactions in Examples 7A and 8A show that ligand VII combined with a methylallylnickel complex decreased C6 olefin production and increased C4 olefin production when propylene is present in a two-fold excess at 0° C. and the reactor pressure is increased from 2.4 atm to 4.4 atm. Ligand VII (Example 8A) increased the mole % of C4 products from 9% to 51% relative to the control example (Example 7A) which uses no phosphine ligand.

### Examples 9A-15A

The reactions in Examples 9A-15A demonstrate the effects of running the co-dimerization experiments in a 1:2 ethylene:propylene atmosphere at room temperature (20° C.) with various ligands mixed with methylallylnickelchloride. The control experiment (Example 9A) used no phosphine ligand. Ligand VII (Example 10A) decreased the amount of C6 olefins from 45% in the control to 22% and concurrently increased the amount of C4s, and C5s produced. While ligand IX (Example 11A) lowered the amount of both the C5 and C6 products while increasing the amount of C4s relative to the control (Example 9A), it also increased the branched C5 selectivity from 18% to 73%. Although the amount of C6 olefins formed is higher than in the control experiment, the increase in branched C5 selectivity was more pronounced with ligands XI, from 18% to 91% (Examples 12A), and ligand XIII, from 18% to 87% (Example 13A). Ligands III and XII (Examples 14A and 15A, respectively) are included to show their effects on the product selectivity.

### Examples 16A-17A

Examples 16A and 17A show the effect of increasing the reactor temperature to 40° C. in the same 1:2 ethylene:propylene atmosphere at 2.4 atm with methylallylnickelchloride. Example 16, the control experiment, used no phosphine ligand while ligand VII (Example 17A) increased the amount of C5 olefins to 60% and the percent of linear C5s in the C5 portion to 87% relative to the control.

### Examples 18A-25A

The reactions in Examples 18A-25A demonstrate the effects of running the co-dimerizations in an equimolar (1:1) mixture of ethylene and propylene at 0° C. at 2.4 atm with methylallylnickelchloride. The control reaction (Example 18A) showed that the absence of a phosphine ligand affords a C4, C5, and C6 product distribution similar to Calculated Example 1. Ligand VI (Example 19A) afforded 82% C4 products and 46% linear and 54% branched C5s. Use of ligand X (Example 20A) produced 85% branched C5 olefins within the C5 portion of the product. Examples 21A to 25A demonstrated the effects on the C4 to C6 product distribution and the ratio of linear to branched C5s with ligands I, II, IV, V, and VIII respectively.

### Examples 26A-28A

Ligands VII (Example 27A) and VI (Example 28A) with methylallylnickel chloride in the presence of a 1:9 ethylene:propylene reaction mixture at 40° C. under 4.4 atm demonstrated their effect on the product distribution when 90% of the reactant gas is propylene. Ligand VII (Example 27A) decreased the amount of C6 olefins formed and increased the amount of C5s combined with C4s by 80%. Ligand VI (Example 28A) decreased the amount of C6 olefins formed even more effectively and increased the amount of C5s combined with C4s by 166%.

TABLE 3
Co-dimerization Reactions

| | Temp. | Pressure | Ethylene: | | Mole % | Mole % | Mole % | % Linear | % Branched |
|---|---|---|---|---|---|---|---|---|---|
| Example | (° C.) | (atm) | Propylene | Ligand | C4s | C5s | C6s | C5s in C5 | C5s in C5 |
| 1A | RT | 2.4 | 1:2 | none | 24% | 19% | 57% | 46% | 54% |
| 2A | RT | 2.4 | 1:2 | VII | 68% | 21% | 12% | 50% | 50% |
| 3A | RT | 2.4 | 1:2 | X | 75% | 21% | 4% | 50% | 50% |
| 4A | 0 | 2.4 | 1:2 | none | 14% | 39% | 48% | 78% | 22% |
| 5A | 0 | 2.4 | 1:2 | VI | 83% | 15% | 2% | 53% | 47% |
| 6A | 0 | 2.4 | 1:2 | X | 8% | 38% | 54% | 22% | 78% |
| 7A | 0 | 4.4 | 1:2 | none | 9% | 43% | 48% | 75% | 25% |
| 8A | 0 | 4.4 | 1:2 | VII | 51% | 36% | 13% | 81% | 19% |
| 9A | RT | 2.4 | 1:2 | none | 11% | 44% | 45% | 82% | 18% |
| 10A | RT | 2.4 | 1:2 | VII | 28% | 51% | 22% | 81% | 19% |
| 11A | RT | 2.4 | 1:2 | IX | 56% | 24% | 20% | 27% | 73% |
| 12A | RT | 2.4 | 1:2 | XI | 4% | 35% | 61% | 9% | 91% |
| 13A | RT | 2.4 | 1:2 | XIII | 5% | 44% | 51% | 13% | 87% |
| 14A | RT | 2.4 | 1:2 | III | 18% | 33% | 49% | 55% | 45% |
| 15A | RT | 2.4 | 1:2 | XII | 3% | 37% | 60% | 66% | 34% |
| 16A | 40 | 2.4 | 1:2 | none | 9% | 47% | 43% | 83% | 17% |
| 17A | 40 | 2.4 | 1:2 | VII | 21% | 60% | 19% | 87% | 13% |
| 18A | 0 | 2.4 | 1:1 | none | 30% | 39% | 32% | 79% | 21% |
| 19A | 0 | 2.4 | 1:1 | VI | 82% | 14% | 4% | 46% | 54% |
| 20A | 0 | 2.4 | 1:1 | X | 19% | 42% | 39% | 15% | 85% |
| 21A | 0 | 2.4 | 1:1 | V | 41% | 32% | 28% | 80% | 20% |
| 22A | 0 | 2.4 | 1:1 | I | 26% | 37% | 38% | 44% | 56% |
| 23A | 0 | 2.4 | 1:1 | IV | 23% | 44% | 32% | 50% | 50% |
| 24A | 0 | 2.4 | 1:1 | VIII | 28% | 35% | 37% | 32% | 68% |
| 25A | 0 | 2.4 | 1:1 | II | 28% | 47% | 26% | 39% | 61% |
| 26A | 40 | 4.4 | 1:9 | none | 1% | 20% | 79% | 80% | 20% |
| 27A | 40 | 4.4 | 1:9 | VII | 5% | 33% | 62% | 82% | 18% |
| 28A | 40 | 4.4 | 1:9 | VI | 13% | 43% | 44% | 56% | 44% |

1. A catalyst solution comprising:
i. an organic complex of nickel;
ii. an alkyl aluminum compound;
iii. a solvent; and
iv. at least one phosphine compound having the formula:
PR¹R²R³ (I)
wherein R¹ and R² each are independently selected from the group consisting of t-butyl, 2-pyridyl, 2,6-dimethoxyphenyl, o-tolyl, cyclohexyl, phenyl, butyl, and adamantyl; and
R³ is selected from the group consisting of 2-pyridyl, 2,6-dimethoxyphenyl, o-tolyl, 2′,4′,6′-triisopropylbiphenyl, 2′-(N,N-dimethylamino)biphenyl, adamantyl, 1-(2,4,6-trimethyl-phenyl)-1H-imidazole, and 1,2,3,4,5-pentaphenyl-1′-ferrocene.

2. The catalyst solution according to claim 1 wherein the organic complex of nickel comprises bis(triphenylphosphine)dicarbonylnickel, methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentylallylnickel chloride, cyclooctenylnickel chloride, cinnamylnickel bromide, phenylallylnickel chloride, cyclohexenylnickel bromide, cyclodecenylnickel chloride, or a combination thereof.

3. The catalyst solution according to claim 2 wherein the solvent comprises alkanes, cycloalkanes, aromatic hydrocarbons, halogenated aromatic hydrocarbons, ethers, or mixtures thereof.

4. The catalyst solution according to claim 3 wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

5. The catalyst solution according to claim 3 wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

6. A catalyst solution comprising:
i. an allylnickel halide catalyst;
ii. an alkyl aluminum compound;
iii. a solvent; and
iv. at least one phosphine compound selected from the following structures:

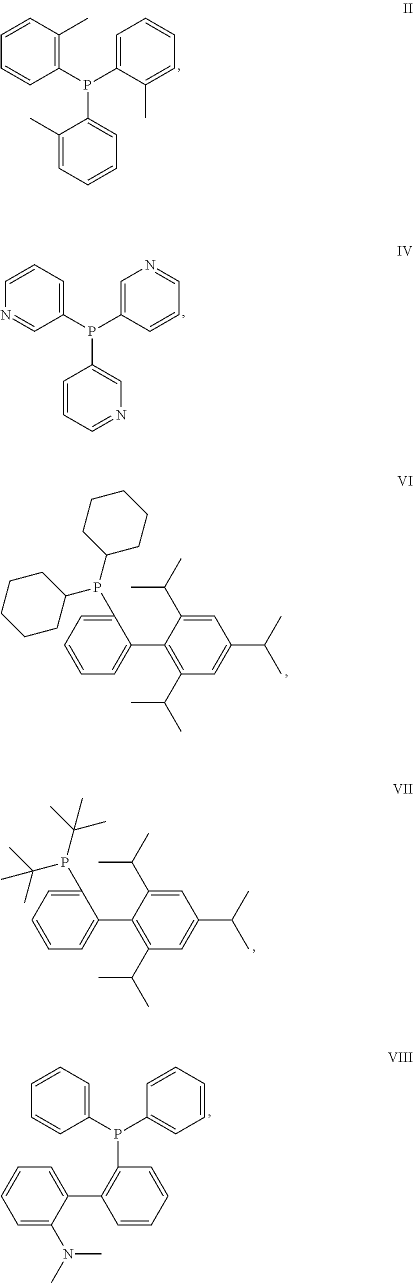

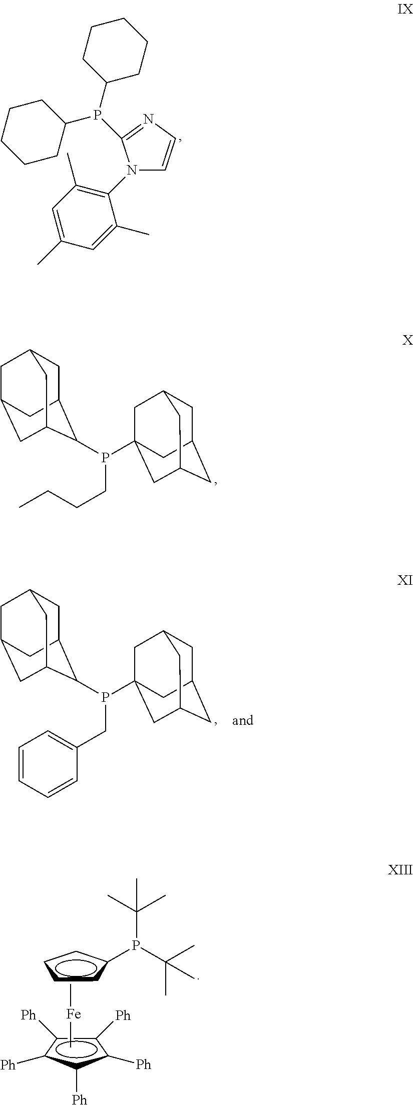

7. The catalyst solution according to claim 6 wherein the allylnickel halide comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentyallylnickel chloride, cyclooctenylnickel chloride, cinnamylnickel bromide, phenylallylnickel chloride, cyclohexenylnickel bromide, cyclodecenylnickel chloride, or combinations thereof.

8. The catalyst solution according to claim 7 wherein the solvent comprises alkanes, cycloalkanes, aromatic hydrocarbons, halogenated aromatic hydrocarbons, ethers, or mixtures thereof.

9. The catalyst solution according to claim 8 wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

10. The catalyst solution according to claim 7 wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

11. An ethylene and propylene co-dimerization process comprising:
contacting ethylene and propylene under elevated pressure with a catalyst solution comprising:
i. an allylnickel halide catalyst;
ii. an alkyl aluminum compound;
iii. a solvent; and
iv. at least one phosphine compound selected from the following structures:

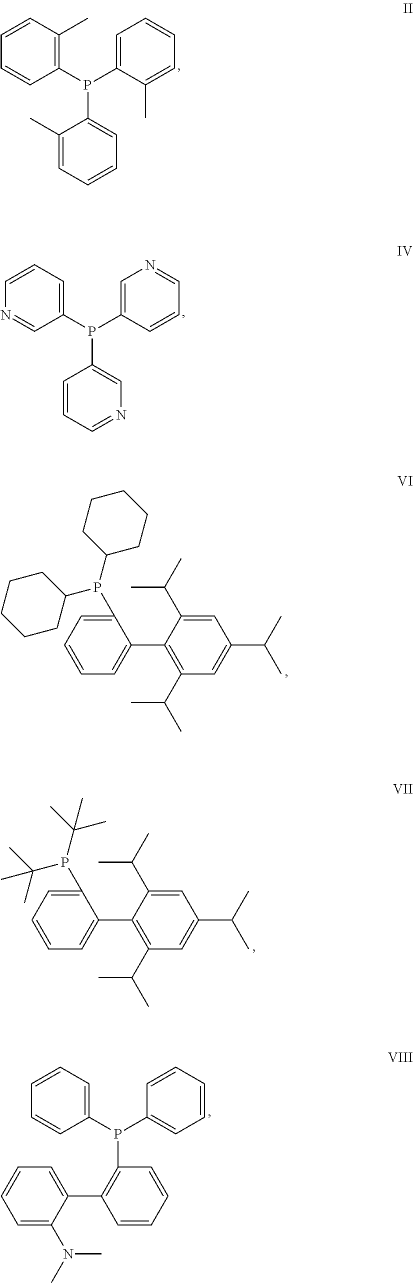

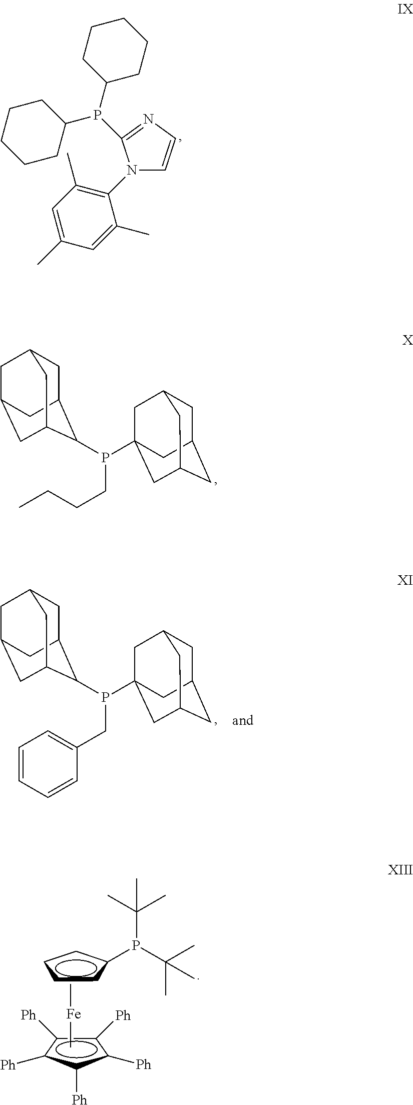

12. The process according to claim 11 wherein the allylnickel halide catalyst comprises methylallylnickel chloride, methylallylnickel chloride dimer, methylallylnickel bromide, methylallylnickel bromide dimer, methyallylnickel iodide, methyallylnickel iodide dimer, allylnickel chloride, allylnickel bromide, allylnickel iodide, crotylnickel chloride, ethylallylnickel chloride, cyclopentyallylnickel chloride, cyclooctenylnickel chloride, cinnamylnickel bromide, phenylallylnickel chloride, cyclohexenylnickel bromide, cyclodecenylnickel chloride, or a combination thereof.

13. The process according to claim 12 wherein the solvent comprises alkanes, cycloalkanes, aromatic hydrocarbons, halogenated aromatic hydrocarbons, ethers, or mixtures thereof.

14. The process according to claim 13 wherein the alkanes comprise dodecane, octane, hexane, heptane, iso-octane mixtures, or a mixture thereof; the cycloalkanes comprise decalin, cyclohexane, cyclooctane, cyclododecane, methylcyclohexane, or a mixture thereof; the aromatic hydrocarbons comprise benzene, toluene, xylene isomers, tetralin, cumene, or a mixture thereof; the halogenated aromatic hydrocarbons comprise chlorobenzene, 1,2-dichlorobenzene, 1,3-dichlorobenzene, or a mixture thereof; and the ethers comprise diethyl ether, dipropyl ether, dibutyl ether, tetrahydrofuran, or a mixture thereof.

15. The process according to claim 14 wherein the alkyl aluminum compound comprises diethylaluminum chloride, methylalumoxane, tri-ethylaluminum, tri-propylaluminum, tri-isopropylaluminum, tri-n-butylaluminum, tri-isobutylaluminum, n-butylaluminum dibromide, ethyl aluminum sesquichloride, methyl aluminum sesquichloride, ethyl aluminum sesquibromide, ethyl aluminum sesquifluoride, or a combination thereof.

16. The process according to claim 15, wherein the molar ratio of ethylene:propylene is about 100:1 to about 1:100.

17. The process according to claim 15, wherein the molar ratio of ethylene:propylene is about 10:1 to about 1:10.

18. The process according to claim 15, wherein the ethylene and propylene are contacted with the catalyst solution at a temperature of about −80 to about 100° C.

19. The process according to claim 18, wherein the ethylene and propylene are added to the catalyst solution wherein the contacting is at a pressure of about 1.5 to about 7 atm.

20. The process according to claim 15, wherein the molar ratio of alkyl aluminum compound to nickel is about 20 to about 1; and wherein the molar ratio of phosphine ligand to nickel is about 1.5 to about 2.
